(19) United States
(12) Patent Application Publication
(10) Pub. No.: US 2004/0229295 A1
(43) Pub. Date: Nov. 18, 2004

(54) Activated delivery of biomolecules using electromagnetic energy

(76) Inventors: Kevin S. Marchitto, Golden, CO (US); Stephen T. Flock, Arvada, CO (US)

Correspondence Address: Benjamin Aaron Adler, ADLER & ASSOCIATES, 8011 Candle Lane, Houston, TX, 77071, US

(21) Appl. No.: 10/774,320

(22) Filed: Feb. 6, 2004

Related U.S. Application Data
(63) Continuation-in-part of application No. 09/573,147, filed on May 17, 2000, now Pat. No. 6,689,380.
(60) Provisional application No. 60/134,486, filed on May 17, 1999.

(51) Int. Cl. G01N 33/53

(52) U.S. Cl. 435/7.5

(57) ABSTRACT

The present invention relates to methods, devices for using electromagnetic energy to inductively alter biomolecular targets for therapeutic or diagnostic purposes. The biomolecular targets optionally may be a composition comprising groups or “sinks” linked to them in order to enhance the magnitude of interaction between the target and the electromagnetic energy.

### CROSS-REFERENCE TO RELATED APPLICATION

This application is a continuation-in-part of non-provisional patent application U.S. Ser. No. 09/573,147, filed May 17, 2000, which claims benefit of provisional application U.S. Ser. No. 60/134,486, filed May 17, 1999, now abandoned.

### BACKGROUND OF THE INVENTION

1. Field of the Invention

The present invention relates generally to the fields of medical physics and drug delivery. More specifically, the invention relates to methods and devices for using electromagnetic energy to inductively alter biomolecular targets for therapeutic or diagnostic purposes.

2. Description of the Related Art

In any given chemical reaction, the equilibrium of the reaction is given by the difference in ΔG⁰ for the reaction. The equilibrium concentrations of substrate (A) and product (C) are determined by their difference in free-energy content,

ΔG⁰=−RTInk_{eq}

where R=gas constant, 8.3 JK⁻¹ mol⁻1, T=absolute temperature and k_{eq}=equilibrium constant for the reaction.

Heat, when added to a reaction, will alter the free energy content of the reaction and therefore will shift the equilibrium of the reaction. It has also been shown that electromagnetic energy, by translating absorbed energy into translational motion, can enhance chemical reactions. However, there is an activation energy barrier between the substrate and product which is given by ΔG′. This ΔG′ represents the change in free energy that must be put into the system to reach the transition state (FIG. 1A). Similarly, the reaction rate is affected by energy of activation, Eₐ, that reflects the amount of energy that must be added to a reaction for the reactants to reach the transition state. The Arrhenius rate equation describes this reaction rate and is given by:

k=Aexp(−Eₐ/RT)

where A=pre-exponential factor and Eₐ=activation energy. Given then, that ln(k)=ln(A)−Eₐ/RT, a plot of ln(k) vs 1/T, gives intercept A and slope −Eₐ/R. If the Eₐ is high, only a portion of the molecular encounters are energetic enough to result in reaction, but, if Eₐ is low, a high proportion can react and the rate coefficient is large. Thus, if the activation energy can be lowered in some way, the reaction proceeds more rapidly.

Anything that stabilizes the transition state relative to reactants will decrease the free energy of activation and therefore increase the reaction rate (FIG. 1B). A catalyst lowers the activation energy of the rate determining steps, thereby speeding up the reaction. The role of the catalyst is to permit the formation of a transition state of lower energy, that is, with higher stability relative to reactants, than that for non-catalyzed reactions. The catalyst itself does not participate in the reaction stoichiometry and, thus, is not consumed, and cannot affect the equilibrium position of the reaction. Pauling expressed that stabilization of the transition state of a reaction by an enzyme suggests that the enzyme has a higher affinity for the transition state than it does for the substrate or products. ΔG′, therefore, is reduced during catalysis.

In order to undergo a chemical reaction, a reactant or reactants must first gain energy, i.e., the activation energy, to form an activated complex before they can proceed forward to a state, products, of different energy or enthalpy. Often, enzymes are used to help the reaction take place, i.e. catalyze the reaction (FIG. 1C). Heat can also be used to speed up the reaction rate. Additionally, vibrational excitation can be used to promote endoergic or energy consuming reactions (2).

Biochemical reaction experiments involving thermal denaturation (1) on human serum albumin show that the protein denatures at 55±3° C. with a total change in free energy for unfolding of 42 kJ·mol⁻1 and the protein aggregates, i.e. clusters together with itself and other proteins, after unfolding. It is also known that the presence of particular chemical species can stabilize or destabilize proteins against thermal denaturation. It is the biochemical reaction of protein denaturation followed by aggregation that is hypothesized to be the underlying mechanism of laser-tissue welding as disclosed in, for example, U.S. Pat. No. 5,713,891. Laser-tissue welding is more fully described in, for example, U.S. Pat. Nos. 6,583,117, 6,391,049, 6,323,037, 6,211,335 and 5,929,044.

The polymerase chain reaction (PCR) is a technique used for in vitro and in situ amplification of specific DNA sequences. The process goes in receptive cycles: denaturing, whereby the DNA of interest is denatured for about 4 minutes at 94° C.; annealing, whereby the appropriate part of the DNA strands are annealed to the primers, i.e., the antisense DNA fragment of interest, at 50° C. for about 2 minutes; and extension, whereby the heat stable enzyme Taq-DNA-polymerase (Taq) polymerizes the individual DNA bases, deoxyribonucleotides, for 3 minutes at 72° C. Such cycle is repeated for N times (−20-30 times) with more primers and nucleotides added. For the following cycles, heating parameters may be modified slightly, for example, denaturing for 1 min. at 94° C.; annealing for 2 min. at 50° C.; and extending for 3.5 min at 72° C. Additionally, the heating parameters for the last cycle also are typically different, for example, denaturing for 1 min. at 94° C.; annealing for 2 min. at 50° C.; and extending for 10 min at 72° C. As a result, the DNA of interest is amplified by 2^{N}.

For the heating protocol example above, the total time for denaturation is 4+(20×1)+1=25 minutes with additional time needed for cooling (if N=20). If it were possible to denature and cool the DNA more efficiently, a significant timesaving would result. Furthermore, the part of the PCR cycle that involves the highest temperatures would be eliminated and thermal breakdown of the reaction buffer materials would be reduced. Further, increasing the reaction rate of the annealing and extension phases of PCR would also add a timesaving.

Inductive heating (3) is a non-contact process whereby electrical currents are induced in electrically conductive materials (susceptors) by a time-varying magnetic field; the current ultimately gives rise to ohmic (Joule) heating. Generally, induction heating is an industrial process often used to weld, harden or braze metal-containing parts in manufacturing where control over the heating process and minimized contact with the work piece are critical.

The theory of induction heating involves radiofrequency power coupled to a conducting element, such as a coil of wire, which serves to set up a magnetic field of a particular magnitude and spatial extent. The induced currents or Eddy currents flow in the conductive materials in a layer referred to as the skin depth δ, given by: δ=√(2ρ/Ωμ),

where Ω is frequency (rads/s), ρ is resistivity (ohm-m) and μ is the permeability (Webers/amp/m) which is the product of μₒ the permeability of free space and μᵣ the relative permeability of the material.

The magnetic permeability of a material is quantification of the degree to which it can concentrate magnetic field lines. Note, however, that the permeability is not constant in ferromagnetic substances like iron, but depends on the magentic flux and temperature. The skin depth of 1 MHz electromagnetic radiation in copper at room temperature is 0.066 mm and in 99.9% iron is 0.016 mm.

The consequence of current flowing is Joule, or I²R, heating. The skin-depth formula leads to the conclusion that, with increased frequency, the skin depth becomes smaller. Thus, higher frequencies favor efficient and uniform heating of smaller components. In certain situations localized heat can also be generated through hysteresis losses or frictional heating, referred to as dielectric hysteresis heating in non-conductors, as the susceptor moves against physical resistance in the surrounding material. Consideration of Joule heating alone results in a formula for the power-density P (W/cm³) in the inductively-heated material:

P=4πH²μfM,

where H is the root-mean-square magnetic field intensity (A/m), f is frequency (Hz), M is a power density transmission factor (unitless) which depends on the physical shape of the heated material and skin depth and diameter of the part to be heated.

M, which is equal to the product of F and d, where F is a transmission factor and d is the diameter of the object inductively coupled to the magnetic field, can be shown to be maximally about 0.2 when the object diameter is 3.5 times the skin depth and when certain other assumptions are made. Thus, for a given frequency there is a diameter for which the power density is a maximum; or equivalent, there is a maximum frequency for heating a part of a certain diameter below which heating efficiency drops dramatically and above which little or no improvement of heating efficiency occurs. It can also be shown that the power density of inductively heated spheres is much higher than solid spheres of the same material.

It is apparent from the current industrial uses of inductive heating, that using this technique to heat macroscopic solid pieces of electrically conducting metal is relatively easy (albeit it requires large and expensive power supplies and solenoid-type coils), but heating small (<1 mm) particles is difficult; in fact, powdered metals are used in some high-frequency transformers as the powder does not inductively heat to any extent and so the transformer stays cool during operation.

Only a few examples of the use of in vivo inductive heating are found in the medical literature. The oldest example of use of therapeutic inductive heating is in hyperthermia of cancer, whereby large metallic “seeds” are inductively heated using a coil external to the body. Smaller seeds consisting of dextran magnetite particles in magnetic fluid have been used to treat mouse mammary carcinoma by hyperthermia (4). Hyperthermia always involves temperatures of about 43° C. that are below the threshold for protein denaturation. U.S. Patent Application No. 2002/0183829 describes inductively heating solid metal stents positioned in diseased blood vessels for the purpose of killing proliferating smooth muscle cells in restenosing blood vessels.

There are few examples in the medical literature of the use of in vitro inductive heating. U.S. Patent Application No. 20020061588 described the use of induction heating to heat nanocrystals coupled to DNA to locally denature DNA for the purpose of hydridization (5). U.S. Patent Application No. 20020119572 describe using an external electromagnetic field to alter the property of a protein to which is attached a susceptor, such as a chromophore or metallic or semi-conducting nanoparticle. The susceptor serves to absorb the ambient electromagnetic field.

U.S. Pat. No. 6,348,679 discloses compositions used in bonding two or more conventional materials where the interposed composition consists of a carrier and a susceptor, which may be at least in part composed of certain proteins. This application applies only to conventional substrates, such as films or wood, so issues such as biocompatibility and extraneous thermal damage are not relevent. U.S. Pat. No. 6,323,037 describes the addition of an optical “energy converter” to the solder mixture such that incident optical energy will be efficiently and preferentially absorbed by the solder which subsequently effects a tissue weld. Similar optical susceptors are described in U.S. Pat. No. 6,530,944.

Drug delivery is a critical aspect of medical treatment. In many cases, correct administration of drugs is critical to the overall efficacy of its action and, thus, patient compliance becomes a significant factor in therapy. For this reason, the physician should carefully monitor drug delivery. Drug delivery is particularly important in acute care settings. Patients must often endure long hospital stays post-surgery or other treatment to ensure that drugs are administered properly. In this case, and in many others, a patient must remain in close contact with the physician during the course of treatment. This compliance issue and the cost of long-term hospital stays have resulted in significant research and development of devices capable of delivering controlled, continuous and sustainable release of therapeutics.

Skin has a very thin layer of dead cells, called the stratum corneum, which acts as an impermeable layer to matter on either side of the layer. The stratum corneum is what primarily provides the skin's barrier function. If the stratum corneum is removed or somehow altered, then materials within the body can more easily diffuse out to the surface of the skin and materials outside the body can more easily diffuse into the skin. Alternatively, compounds referred to as permeation enhancers, e.g. alcohol, or drug carriers, e.g. liposomes, can be used, with some success, to penetrate the stratum corneum. In any case, the barrier function of the skin presents a very significant problem to pharmaceutical manufacturers who may be interested in topical administration of drugs or in transcutaneous collection of bodily fluids.

Mucosa, which is the moist lining of many tubular structures and cavities, such as the nasal sinuses and the mouth, consists in part of an epithelial surface layer. This surface layer consists of sheets of cells with strong intercellular bonds, in single or multiple layers, and has a non-keratinized or keratinized epithelium. On the basolateral side of the epithelium is a thin layer of collagen, proteoglycans and glucoproteins called the basal lamina which serves to bind the epithelial layer to the adjacent cells or matrix. The mucosa acts as a barrier to prevent the significant absorption of topically applied substances, as well as the desorption of biomolecules and substances from within the body. The degree to which mucosa acts as a barrier and the exact nature of the materials to which the mucosa is impermeable or permeable depend on the anatomical location. For example, the epithelium of the bladder is 10,000 times less “leaky” to ions than the intestinal epithelium.

The mucosa is substantially different from skin in many ways. For example, mucosa does not have a stratum corneum. Despite this difference, permeation of compounds across mucosa is limited and somewhat selective. The most recent model of the permeability of mucosa is that the adjacent cells in the epithelium are tightly bound by occluding junctions that inhibit most small molecules from diffusing through the mucosa while allowing effusion of mucoid proteins. The molecular structure of the epithelium consists of strands of proteins that link together between the cells, as well as focal protein structures such as desmosomes.

The permeation characteristics of mucosa are not fully understood, but it is conceivable that the selective permeability of the mucosa may depend on this epithelial layer, which may or may not be keratinized, as well as the basal lamina. While it has been shown that removal or alteration of the stratum corneum of skin can lead to an increase in skin permeability, there is no corresponding layer on the mucosa to modify. Thus, it is not obvious that electromagnetic energy irradiation will cause a modification of the permeability of mucosa.

Various methods have been used for facilitating the delivery of compounds across the skin and other membranes. Iontophoresis uses an electric current to increase the permeation rate of charged molecules. Iontophoresis however is dependent on charge density of the molecule and, furthermore, has been known to cause burning in patients. Use of ultrasound also has been tested whereby application of ultrasonic energy to the skin results in a transient alteration of the skin thereby increasing its permeability to substances. U.S. Pat. No. 4,775,361 discloses that electromagnetic energy produced by lasers may be used to ablate stratum corneum in order to make the skin more permeable to pharmaceutical substances. In U.S. Pat. No. 5,614,502 impulse transients generated by lasers or by mechanical means may be used to make alterations in epithelial layers that result in improved permeation of compounds.

There are many therapeutic and diagnostic procedures that would benefit from a transmucosal or transendothelial route of administration or collection. For example, local anesthetics, such as lidocaine, are delivered to a region prior to a medical treatment. Such a local administration of lidocaine could be efficacious at providing anesthesia, but would minimize any side-effects and eliminate the need for a needle. Local administration of an antineoplastic drug into the bladder wall could greatly minimize the time required for a patient to hold a drug in the bladder during chemotherapy.

Electrosurgery is a method whereby tissue coagulation and/or dissection can be effected. In electrosurgery radiofrequency (RF) current is applied to tissue by an active electrode. In a bipolar system, the current is passed through tissue between two electrodes on the same surgical instrument, such as a forceps. In a monopolar system, a return-path or ground electrode is affixed in intimate electrical contact with some part of the patient. Because of the importance of the ground electrode providing the lowest impedance conductive path for the electrical current, protection circuits monitoring the contact of the ground with the patient are often employed whereupon an increase in ground electrode-skin impedance results in the instrument shutting down. Factors involved in an electrosurgical system include treatment electrode shape, electrode position, i.e., contact or non-contact, with respect to the tissue surface, frequency and modulation of the RF, power of the RF, and time for which it is applied to the tissue surface, peak-to-peak voltage of the radiofrequency and tissue type.

In typical electrosurgical systems, radiofrequency frequencies of 300 kHz to 4 MHz are used since nerve and muscle stimulation cease at frequencies beyond 100 kHz. For example, all else being equal, decreasing electrode size translates into increased current density in the tissue proximal to the electrode and so a more invasive tissue effect, such as dissection, as compared to coagulation. Similarly, all else being equal, if the electrode is held close to the tissue, but not in contact, then the area of RF-tissue interaction is small, as compared to the area when the electrode is in contact with the tissue, so the effect on the tissue is more invasive.

By changing the waveform of the applied RF from a continuous sinusoid to packets of higher peak voltage sinusoids separated by dead time, i.e. a duty cycle of, say, 6%, then the tissue effect, again with all else being equal, can be changed from dissection to coagulation. Holding all else equal, increasing the voltage of the waveform increases the invasiveness of the tissue effect. Of course, the longer the tissue is exposed to the radiofrequency, the greater the tissue effect. Finally, different tissues respond to radiofrequency differently because of their different electrical conductive properties, concentration of current carrying ions and different thermal properties.

The prior art is deficient in the lack of inductively adding energy to biomolecules and/or biomolecules enhanced to interact with the electromagnetic energy to accelerate the rate of a biochemical reaction in vitro or in vivo. More specifically, the prior art is deficient in the lack of effective means of controlling the rate of pharmaceutical delivery or biomolecule collection by utilizing inductive electromagnetic energy. The present invention fulfills these long-standing needs and desires in the art.

### SUMMARY OF THE INVENTION

The present invention describes methods and devices for “activated delivery” of biomolecules, for example, macromolecules, such as proteins, carbohydrates and lipids, including bioactive molecules, such as, but not limited to, pharmaceuticals, biologics, biomaterials, for example, any non-drug material that can be used to treat, enhance, or replace any tissue, organ or function in an organism. These molecules are modified through the addition of energy absorbing structures onto the molecules, which improves their reactivity upon exposure to an electromagnetic energy source. The absorbed energy is most likely transduced to heat or kinetic energy, resulting in increased excitation of the molecules, in turn resulting in increased migration of the molecule, or of its molecular groups. The energy source may be, for example, a laser, or a radiofrequency power supply.

The present invention is directed to a composition comprising at least one biomolecule and an electromagnetic energy absorbing species associated therewith. The absorbing species may be associated via a chemical linker, one or more chemical bonds or via a physical association, such as diffusion.

The present invention also is directed to a related composition comprising at least one biomolecule and a susceptor. The susceptor may be associated with the biomolecule(s) as described supra.

The present invention is directed further to a method for increasing the energy of biomolecules. The biomolecules are associated with an energy absorbing substance to form the composition described herein. Electromagnetic energy is applied to the composition such that the electromagnetic energy absorbed by the absorbing species is transferred to the biomolecule(s) thereby increasing the energy thereof.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1A depicts reaction coordinates as a function of the free energy.

FIG. 1B shows reaction coordinates as a function of the free energy in the presence of a catalyst.

FIG. 1C shows reaction coordinates as a function of the free energy in the presence of an enzyme. The intermediate transition state (1) and (2) are indicated.

FIG. 2 shows a transdermal patch with electromagnetic energy controller, electrode for transmitting energy, drug reservoir containing the formulation and adhesive backing for attachment to skin.

FIGS. 3A-3D depict different ways to position an inductive transducer in proximity to the reactant.

FIG. 4 is a diagram depicting conformational changes in a protein upon denaturation and aggregation.

FIG. 5A shows device used for enhancing a PCR reaction. FIGS. 5B-5E show that different shaped radiant energy absorbing targets within the walls of a reaction chamber in the device in 5A, or inside the reaction solution, produce different pressure waves.

FIG. 6 is an energy diagram demonstrating alternating cycles of radiant energy for multi-step reactions such as the optically enhanced PCR. Energy reaching level (2) is sufficient to separate the strands of DNA. The energy is then reduced to a lower level (1) that favors a second reaction in the sequence by catalysis. Level (1) is not great enough to denature the molecular products. This cycle is repeated to produce long strands of DNA.

FIGS. 7A-7D depict different arrangements of an induction applicator positioned in proximity to a reaction chamber (FIGS. 7A-7B) and to a multiwell screening plate (FIGS. 7C-7D) in order to enhance in vitro biochemical reaction rates.

FIG. 8 shows a coil type applicator, substantially made out of an electrically non-conducting material, positioned on the arm of a subject. The coil inductor antenna is housed within the applicator. This device could be used in vivo to induce conformational changes in reactants coupled with transducer species.

FIG. 9A shows a sheep artery 90, dissected through the lumen and then anastomosed at 92, by accelerating the biochemical reaction of protein denaturation and aggregation. FIG. 9B shows the histologic section of a sheep carotid artery shows the region 96 where the two vessel ends have been joined and which has nickel transducer flakes present.

FIG. 10 compares temperature over time for heating compositions using a commercially available induction power supply.

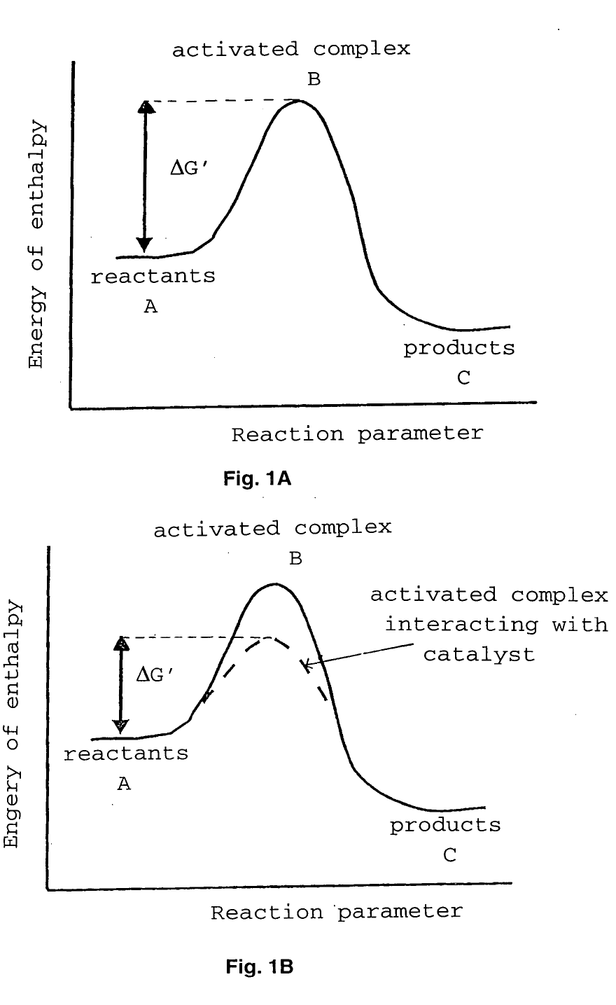

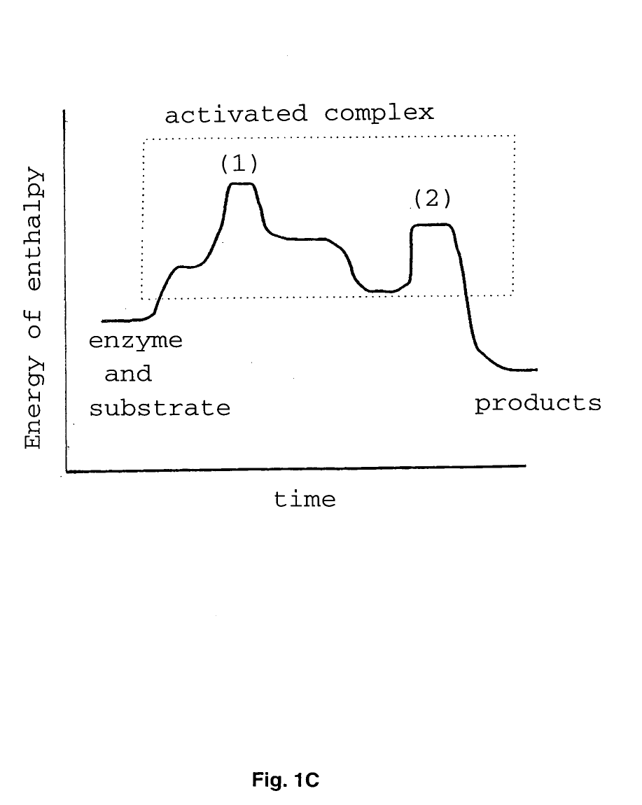

### DETAILED DESCRIPTION OF THE INVENTION

In one embodiment of the present invention there is provided a composition comprising at least one biomolecule and an electromagnetic energy absorbing species associated therewith. Further to this embodiment the composition may comprise a liposome incorporating the composition therein.

In aspects of this embodiment the biomolecule may be associated with the electromagnetic energy absorbing species via a chemical linker. An example of a chemical linker is an avidin/biotin linker. The biomolecule may be associated with the electromagnetic energy absorbing species via a chemical bond. In another aspect the biomolecules form a dimer via a chemical bond whereby the dimer is associated with the electromagnetic energy absorbing species via the same chemical bond or via a different chemical bond. In yet another aspect the biomolecules are associated with the electromagnetic energy absorbing species via a physical process. An example of a physical process is diffusion.

In these aspects the biomolecule, the electromagnetic energy absorbing species or both undergo a change in state upon application of electromagnetic energy to the composition. Representative examples of a change in state are denaturation or a cleaved bond. The electromagnetic energy absorbed by the absorbing species may be laser generated or radiofrequency energy. The electromagnetic energy may be inductively applied to the absorbing species.

In all aspects the biomolecule may be a protein, a carbohydrate, or a lipid or a combination thereof. The biomolecule also may be a pharmaceutical, a biologic, a biomaterial, or a diagnostic or a combination thereof. The electromagnetic energy absorbing species may be a susceptor. Such a susceptor may be a metal. The susceptor also may form a dipole. Alternatively, the electromagnetic energy absorbing species may be a dye.

Furthermore, in all aspects the electromagnetic energy absorbing species may comprise matter with non-zero electrical conductivity. The matter may be diamagnetic, paramagnetic, or ferromagnetic. The matter may be an ionomer, a conducting polymer, an alkali metal, a transition metal, a lanthamide, or a metalloid or a combination thereof. The matter may be a metal nano- or micro-particle, a semiconducting nano- or micro-particle, a magnetic nano- or micro-particles, a polystyrene encapsulated metal particle, a buckminsterfullerene, or liposome encapsulated metal particles.

More specific examples are colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, tungsten, calcium salts, magnesium salts, or alloys of materials from this group.

In a related embodiment there is provided a composition comprising at least one biomolecule and a susceptor associated therewith. In an aspect of this embodiment the biomolecules comprise at least one protein and the composition further comprises a liposome with the protein(s) and the susceptor incorporated therein. Further to this aspect the composition may comprise a pharmaceutical incorporated into the liposome.

In aspects of this embodiment the biomolecule may be associated with the susceptor chemically or physically as described supra. Additionally, in all aspects the biomolecules and the changes in state undergone by the biomolecule(s) and/or the susceptor, as described supra.

Furthermore, in all aspects of this embodiment, the susceptor may be a metal, a metal nano- or micro-particle, a semiconducting nano- or micro-particle, a magnetic nano- or micro-particles, a polystyrene-encapsulated metal particle, a buckminsterfullerene, or liposome encapsulated metal particles. Examples may be colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, tungsten, or alloys of materials from this group.

In another embodiment of the present invention there is provide a method for increasing the energy of biomolecules comprising the steps of associating the biomolecules with an energy absorbing substance to form the composition described supra; and applying electromagnetic energy to the composition wherein the electromagnetic energy absorbed by the absorbing species is transferred to the biomolecules thereby increasing the energy thereof. Further in this embodiment the method comprises accelerating a biochemical reaction having the biomolecules as reactants via the increase in energy.

In an aspect of this embodiment the biochemical reaction results in a conformational change in the biomolecules. An example of a biochemical change is denaturation. In another aspect the biochemical reaction may be enzyme catalyzed. Examples of enzyme-catalyzed biochemical reaction are a polymerase chain reaction or an enzyme-linked immunosorbent assay. In all aspects the biomolecules may be present in tissue or may be in vitro.

In all aspects of this embodiment the electromagnetic energy may be radiofrequency energy. The electromagnetic energy may have a frequency from about 100 kHz to 40 GHz. Preferably, the electromagnetic energy is from about 400 kHz to 2.4 GHz. Alternatively, the electromagnetic energy may generate a magnetic field.

The present invention provides methods/devices for remote and local controlled delivery of pharmaceutical compounds or collection of biomolecules using electromagnetic energy. Electromagnetic devices described herein, including laser systems, are used to effect drug delivery and may be controlled locally or remotely by microprocessors integrated into the devices, which are in turn programmed to receive or transmit data over telecommunication networks. Controlled electromagnetic energy driven systems may be integrated into patches and other delivery devices that may be worn on the skin, or may be implanted. Electromagnetic energy may be light or radiative energy, radiofrequency energy or microwave energy.

These devices may contain microprocessors or other electronically controlled elements that can be interrogated remotely or locally using integrated or remote transmitters. The transmitters in turn may be communicated with via telecommunication networks. Equipment used in paging systems is built into a transdermal patch that receives a signal, or code, sent by the operator over telecommunication networks. Control may also be exerted by coding systems, including bar and magnetic coding, communicated by devices that generate and read such code. Code may also be communicated via the Internet, either directly in the case of visible code, or through the use of dedicated communications devices that receive and process code.

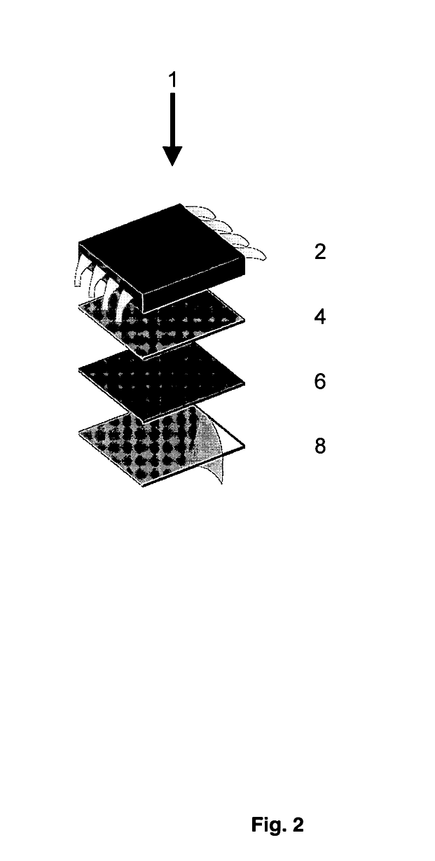

The pharmaceutical delivery device or the biomolecule collection device may be incorporated into a patch. Patches, such as transdermal drug delivery patches, are controlled by remote or local operation through microprocessors. Such patches are designed and used together with electromagnetic energy driven delivery systems. The dressing is in contact with an electrode, which is, in turn, in contact with the controller. The controller regulates the flow of electromagnetic energy that contacts the electrode. The electrode distributes the energy to the pharmaceutical, and further into the tissue of interest. Therefore, as depicted in FIG. 2, a transdermal patch 1 comprises an electromagnetic energy controller 2, an electrode for transmitting energy 4, drug reservoir 6 containing the pharmaceutical or other composition and adhesive backing 8 for attachment to skin (not shown).

Patches used herein include a dressing material which contains a gel or adhesive that in turn contains the drug formulation to be delivered. Besides a dressing, the patch may be a gel, viscous material, or other patch material that covers the site of treatment. Pharmaceuticals such as, but not limited to, an anesthetic drug, an anti-neoplastic drug, a photodynamic therapeutical drug, an anti-infection drug, and an anti-inflammatory drug may be used in the patch. For example, the anesthetic drug is lidocaine.

The patch may contain one or more reservoirs. In the case of multiple reservoirs, a rupturable membrane may separate different chambers, thus preventing mixing of components until the membrane is ruptured. In the case of an unstable compound such as prostaglandin E1 (e.g. Caverject, UpJohn), lyophilized crystals could be stored in one reservoir while the liquid components to be mixed with the drug could be stored in another reservoir. The two reservoirs are separated by a membrane that may be ruptured by crushing or other physical means, thereby allowing the components to mix freely to make available for dosing. This multi-reservoir concept may be further extended to include mixing of chemicals that will generate an electrical current, for the purpose of iontophoresis or electroporation.

Healing at the site of ablation will ultimately reduce the amount of drug that permeates over time. A substance may be included in the drug formulation or patch and applied to the site of permeation/ablation whereby this substance slows the healing process or reduces the rate of scab formation thereby limiting the rate of closure of the permeation site and having the effect of extending the enhanced permeability characteristics of the irradiated site.

The controller may be comprised of a current generator driven by a transportable battery, a solar powered generator, an electrochemical generator, a thermal energy generator, a piezoelectric generator, a radiofrequency generator, a microwave generator, etc. The electrode of the patch may be replaced by a laser, multiple lasers, or optical fibers which conduct and transmit light at a desired wavelength, pulse length, pulse energy, pulse number and pulse repetition rate to ablate or alter the tissue directly beneath the patch. Alternatively, continuous wave lasers may be used to effect alterations in the tissues that would lead to a permeabilizing effect. Lasers and other electromagnetic energy generators, as well as ultrasound transducers, may be used in controller-electrode combinations that will result in the desired effects. Also inherent in the design of these patches is the ability to deliver the drugs simultaneously with the energy, or at any time before or after energy administration.

Telecommunication networks transmit data between the operator and the remotely sited controller on the patch. The device includes a telemetry transceiver for communicating data and operating instructions between the device and an external patient communications control device that is either worn by, located in proximity to the patient, or at a remote location within the device transceiving range. The control device preferably includes a communication link with a remote medical support network through telecommunication network(s) and may include a global positioning satellite receiver for receiving positioning data identifying the global position of the control device.

The device may also contain a patient activated link for permitting patient initiated personal communication with the medical support network. A system controller in the control device controls data and digital communications for selectively transmitting patient initiated personal communications and global positioning data to the medical support network, for receiving telemetry out of data and operating commands from the medical support network, and for receiving and initiating re-programming of the device operating modes and parameters in response to instructions received from the medical support network. The communication link between the medical support network and the patient communications control device may comprise a world wide satellite network, hard-wired telephone network, a cellular telephone network or other personal communication system.

An objective of the device is to provide the patient greater mobility. The patient is allowed to be ambulatory while his medical condition is monitored and/or treated by the medical device. Programming devices may be controlled by the physician or pharmacy, and code or data transmitted over telecommunication networks to the site of the device. This can happen remotely or locally. Currently, telemetry systems used to communicate with medical devices are positioned within a short distance of the device. Furthermore, transdermal patch systems are currently regulated only by passive controls, built into the patch, which regulate the dosage. These controls are typically not electronic, but rather based on membrane and diffusion characteristics of the patch and formulation. The present device provides a means for a remote operator to adjust the dosage and timing of drug delivery, while the patient is ambulatory.

Another object of the device is to provide a patient data communication system for world-wide patient re-programming telemetry with a medical device worn by the patient. The device described herein is a transdermally worn patch containing a drug formulation which may or may not include electromagnetic energy permeation enhancement. However, the invention is not limited to transdermal drug delivery and may include communication with implanted drug delivery devices using the aforementioned electromagnetic energy based delivery technology.

The presently disclosed device transmits and receives coded information from a remote or local source. The operator at the device is positioned at a location, and can transmit information from the medical support network. The device incorporates a wireless interface including a control device telemetry transceiver for receiving and transmitting coded communications between the system controller and the device telemetry transceiver, a global positioning system coupled to the system controller for providing positioning data identifying the global position of the patient to the system controller, communication means for communicating with the remote medical support network, and communication network interface means coupled to the system controller and the communication means for selectively enabling the communication means for transmitting the positioning data to the medical support network and for selectively receiving commands from the medical support network.

The communication interface may include capabilities for transfer of data between the patient and the operator by cellular telephone network, paging networks, satellite communication network, land-based telephone communication system, or modem-based communication network, including a computer monitor connected to the Internet. Communications may include but not be limited to microwave, radiofrequency and digital communication via optical means.

The communication and monitoring systems provide a means for exchanging information with and exercising control over one or more medical devices attached to the body of a patient. The devices are intended to function no matter how geographically remote the patient may be relative to the monitoring site or medical support network. The operator, usually a physician, types in a code, which is transmitted over the medical support network to the patient, who may be located by a geopositioning satellite or in relation to other telecommunication network. The code contains information which activates the device and controls dosage.

Dosage schedules for certain medications can be pre-encoded by the manufacturer or pharmacy, using bar code symbols. The encoded bar code symbols can be compiled on one or more menu sheets accessible at the physician office or pharmacy counter where the controller is installed. In such applications, a bar code symbol-reading device can be linked to a data communication port of the medical support network and located on the patch, which can then be used to program the proper dosage into the device. The code could be transmitted over the Internet, or via other telecommunication networks into a device in the possession of the patient, who can then read the bar code into the patch.

Data transmission to and from the operator to the device is accomplished by means of a control device that transmits data over the communication network. A telemetry antenna and associated transmitter/receiver can both download and upload data. The antenna may function on radiofrequencies. Control of dosage in the device itself is provided by a digital controller/timer circuit with associated logic circuits connected with a microcomputer. The microcomputer controls the operational functions of a digital controller and a timer. It specifies activation, timing and duration of events. The microcomputer contains a microprocessor and associated RAM and ROM chips, depending on the need for additional memory and programmable functions.

A base station may exist at the operator's location. The base station may be comprised of a microprocessor-controlled computer with hardware and software, and associated modem for transmission of information that is relayed through the appropriate communication network. The system controller may also be coupled to a GPS receiver for receiving positioning data from an earth satellite. The GPS receiver may use currently available systems.

The present study provides methods and means by which molecules may be propelled through a medium at differential rates relative to the medium and other molecules in the medium, and a means by which molecules may be separated from one another based on their optical characteristics. As such, the present invention provides methods for driving compounds through skin or membranes. Also, the present invention provides methods of altering the barrier function of tissue or membranes or of creating pores in skin. Furthermore, the present invention provides means to move molecules along an optical trap.

Pressure waves created through the interaction of electromagnetic energy with tissue or non-biological matter may be used to drive molecules in a medium across tissue interfaces or between cellular junctions such as those found in membranes, between cells, or even through cellular membranes. The interaction of radiofrequency or microwave irradiation with tissue or another absorber and various pharmaceutical formulations can lead to the generation of propagating pressure waves that are generated from a rapid volumetric change in the medium by heating or by the generation of plasma. The propagating pressure waves are in the form of low pressure acoustic waves propagating at the speed of sound or high pressure shock waves propagating at supersonic speeds. These waves can also be a consequence of a generation of waves in a non-biological target that is in intimate acoustic contact with the biological media.

Continuously pulsing electromagnetic energy delivered in discrete short duration pulses propagates the pressure waves which thereby physically move the molecules between cellular junctions or across membranes. The “pumping” effect may occur through the creation of increased pressure, including osmotic or atmospheric pressure. A separation results which is due to the differential resistance of the tissues or membranes relative to the fluid medium, which is the mobile phase. The degree of pumping will be related to the shape, duty cycle and power of the driving RF. Pumping may at times be inefficient if the energy is deposited directly on a tissue due to its large surface area. To compensate for this inefficiency, a target material, which preferentially absorbs energy at these radiofrequency frequencies, may be placed adjacent to the tissue, in order to transfer energy effectively. This target could effectively act as an antenna and may optionally be composed of metals or metal containing compounds.

Pressure waves can be used to alter the skin or membrane itself thereby reducing its barrier function. This barrier function alteration will be transient; the integrity of the barrier function will reestablish itself soon after the radiofrequency energy ceases to impinge on the tissue. The degree to which the barrier function is reduced will be dependent on the frequency and intensity of the radiofrequency radiation. The pharmaceutical to be applied to the tissue is preferentially in place during irradiation.

The present invention also is used to create pores in skin or membranes or to ablate or alter membranes and tissues. Small pores are made in the skin or membrane by applying the electromagnetic energy with needle-like probes. For example, a patch-like device with thousands of tiny, needle-like probes which conduct electomagnetic energy can deliver the energy to create pores. These probes can be made of silicon with a metallic conducting material.

Radiofrequency or microwave energy is applied directly to the surface of the tissue, or to a target adjacent to the tissue, in such a way that the epithelial layers of the tissue are altered to make the layers “leaky” to substances such as pharmaceuticals. In the case of skin, the stratum corneum may be ablated through the application of electromagnetic energy to generate heat. Alternatively, shear forces may be created by targeting this energy on an absorber adjacent to the skin, which transfers energy to create stress waves that alter or ablate the stratum corneum.

Specifically, radiofrequencies producing a desired rapid heating effect on stratum corneum result in an ablative event, while minimizing coagulation. The removal of the stratum corneum in this way will result in increased permeability of compounds across the compromised tissue interface. For example, application of 4% lidocaine to a section of skin with stratum corneum ablated in this way will result in a rapid, i.e., minutes to onset, anesthesthetic effect.

Alternatively, delivery of electromagnetic energy at these wavelengths may be optimized, by adjusting pulse duration, dwell time between pulses, and peak-power to result in a rapid, intermittent excitation of molecules in the tissues of interest, such that there is no net coagulation effect from heating, but molecules are altered transiently to effect a transient change in membrane conformation that results in greater “leakiness” to substances such as pharmaceuticals. Furthermore, energy with the appropriate pulse mode characteristics is continuously applied that these transient alterations are maintained during the energy cycle, thus creating a means for maintaining increased membrane permeability over time. This method allows substances to be continually delivered over a desired period of time.

The techniques disclosed herein may be combined to effect molecular delivery or collection. For example pressure may be applied to permeabilized membranes. A “leaky” membrane or ablation site in skin is created by first applying electromagnetic energy, including light, microwave or radiofrequency, such that membrane or intramembrane structures are realigned, or the membrane is compromised otherwise, so as to improve permeation. The application of electromagnetic energy induced pressure will drive molecules across tissue interfaces and between cellular junctions at a greater rate than can be achieved by either method alone. The laser energy may be delivered continuously or in discrete pulses to prevent closure of the pore.

Optionally, a different wavelength laser may be used in tandem to pump molecules through the pore than is used to create the pore. Alternatively, a single laser may be modulated such that pulse width and energy vary and alternate over time to alternately create a pore through which the subsequent pulse drives the molecule.

Alternatively, intact skin is treated such that the stratum corneum is compromised leading to a decrease in resistance and increased permeability to molecules in general. As described, application of a electromagnetic energy generated pressure wave will drive molecules across membranes and between cellular junctions at a greater rate than can be achieved by either method alone. Laser energy is directed through optical fibers or guided through a series of optics such that pressure waves are generated to come in contact with or create a gradient across the membrane surface. These pressure waves may be optionally used to create a pressure gradient such that the pressure waves move through a liquid or semi-solid medium thereby “pumping” compounds through the medium, into and across the membrane.

This technology may optionally be used to deliver laser energy for the purpose of drug delivery across, for example, buccal, uterine, intestinal, urethral, vaginal, bladder and ocular membranes. Pharmaceutical compounds may be delivered into cellular spaces beyond these membranes or into chambers encompassed by these membranes. Compromised or intact stratum corneum also may be breached by applying appropriate optical pressure. Furthermore, these methods may be used to control systems that drive substances across non-biological membranes and films. The devices also could be used to increase the diffusion of the substances, as well as endogenous biomolecules, out of tissue.

The present invention also provides methods of using dipole trapping to move molecules, such as pharmaceuticals, contained in a composition or formulation The force arising from a coherent interaction with light is also called the dipole force. A laser field polarizes the atom, and the polarized atom experiences a force in the gradient of an electromagnetic filed. The strong electric filed of a laser beam can be used to induce a dipole moment in a process called dipole or optical trapping. As long as the frequency of the laser field is below the natural resonance of the particle being trapped (e.g. below the atomic transition of an atom or the absorption edge of a polystyrene sphere), the dipole moment is in phase with the driving electrical field. Because the energy of the induced dipole p in the laser field is given by W=−pE; the particle achieves a lower energy state by moving into the high-intensity focal spot of the laser beam. There have been numerous reports of optical traps being used to manipulate particles, or even cells. These traps are used to move these tiny particles around under a microscope lens for manipulation.

Optical tweezers have also been described whereby a focal spot of a single beam optical trap is moved with mirrors or lenses. In the present study, a dipole is formed using radiofrequency or microwave energy, rather than laser energy. The trap is formed at the interface between molecules in a formulation and a tissue or membrane interface. This trap is then moved vectorially in the desired direction of movement. In the case of drug delivery, the desired direction is into the tissues. Thus, the focal point of the trap is moved along a vector that penetrates the tissue of interest, while a formulation containing the drug is applied to the surface of the tissue. The focal point of a single beam or multiple beam trap would then be moved progressively into the tissue, which could occur cyclically so as to ensure the maximum pumping effect.

The present invention also provides methods and devices for enhancing drug delivery or biomolecule collection through membranes by activation of the molecules through the addition of electromagnetic energy. The “activated drug delivery” is further enhanced through modification of the molecules with energy absorbing species. One means of performing the activation is by inductively adding energy.

Specific formulations are chosen such that electromagnetic energy absorption is maximized relative to the surrounding medium such that they become activated upon exposure to an energy source. This may be accomplished through the addition of electromagnetic energy absorbers to the formulation or simply by including them in the surrounding medium.

Generally, biomolecules, including otherwise bioactive molecules, which are naturally occurring in a living organism or those which can have an influence on molecules in a living organism may be used in the formulations and compositions described herein. Typically, such molecules may be found in or around cells and tissues or may be supplied to living organisms, cells and tissues to achieve a desired effect or response. Examples of biomolecules include proteins, carbohydrates or lipids found in cells or tissues. The biomolecules may be, although not limited to, structural such as tissue structures comprising elastin or collagen or structural cellular components such as actin, myosin, or ribonucleoprotein particles. The biomolecules may be involved in catalysis, e.g. as enzymes, or may be reactants such as protease susceptible proteins, metabolized lipids. Examples of other bioactive molecules include, but are not limited to, biological response modifiers, antigens, protease inhibitors, other enzymes, and metabolic inhibitors.

For example, drugs or pro-drugs, biologics, diagnostics or other molecules, including proteins, may be modified through the addition of electromagnetic energy absorbing species or, alternatively, by selecting those that minimize absorption, to maximize the effects of the electromagnetic energy on the formulation itself. In this manner, a new class of compounds is therefore defined that have unique permeability, migration and deposition characteristics as a result of the addition of electromagnetic energy absorbing groups that function in the presence of, or following a treatment of electromagnetic energy as described herein.

These molecules possess different characteristics by virtue of the addition of groups or structures that absorb energy in a characteristic way. The compounds and formulations are designed to include both physiologically active groups and molecular groups which maximize the absorbance or reflectance of energy to achieve the desired effect. Activation of the molecules through the addition of electromagnetic energy results in further modification of the molecules, or in an enhanced ability to interact with molecules in the surrounding medium.

One result is that energy may impart momentum to the altered molecule causing it to move relative to the medium in which it is contained or applied energy may result in excitation of the molecule to result in a further change in that molecule. Upon activation, the molecules adhere, bond, or cross-link with molecular species in the surrounding environment. For example, rapid heating of a molecule, which preferentially absorbs energy relative to its environment, by radiant, radiofrequency or microwave energy, could result in direct activation of a specific activity, in the formation of a bond between the activated molecule and components of the surrounding medium or in the cleavage of a heat-sensitive linkage that results in the release of an active moiety.

An analogy is drawn to pro-drugs which release an active drug upon cleavage, usually enzymatically. An additional analogy is drawn to photodynamic therapy whereby molecules absorb photons, resulting in a transition from ground to an excited singlet state, followed by the transfer of energy to ground state oxygen in the nearby environment, whereupon the oxygen is excited to the singlet state, commonly known as ozone, which is toxic to cells.

In the present example, pharmaceutically active compounds may be modified by the addition of groups that readily form a dipole or serve as energy “sinks” such that localized currents are induced when exposed to appropriate electromagnetic energy, such as radiofrequencies or microwaves. The addition of such dipole-forming groups would result in enhanced ability to use optical trapping methods for the delivery of these types of compounds as described herein. Generally, the addition of such groups would result in enhanced molecular vibration and/or migration of intramolecule electrons, that may further weaken bonds in the modified molecule, or may result in a structural change to that molecule.

Alternatively, carriers selected act as “sinks” for the energy, results in the energy being absorbed preferentially to the sink, thereby limiting exposure to the functional groups to avoid thermal or electronic disruption. Alternatively, molecules may be developed that have functional groups attached to a backbone molecule that is susceptible to cleavage when exposed to electromagnetic energy described herein. Specifically, radiofrequency waves may result in excess vibration of groups as they absorb the energy. Using a linker that is susceptible to cleavage when its atoms vibrate in this way will result in the release of the functional group of interest, which could be a pharmaceutically active substance.

The application is not limited to delivering pharmaceuticals. Other separations of molecules may be achieved by the methods described herein, such as separating protein species in polyacrylamide gels, or separating oligonucleotides on microarray devices. These examples also include using magnetic fields alone to propel molecules through a medium or tissue based on intrinsic magnetic properties or by the addition of magnetic groups, metals, etc. Such methods may also be enhanced by using them in combination with methods to alter membranes and tissues to work synergistically. Further, any compound which may interact with electromagnetic energy in such a way that it is propelled through a medium can be used.

Methods of activated drug delivery may use formulations chosen to effect deposition of a drug or a pool of drugs in a desired region of tissue or cells and to increase the lifetime of the desired species in the tissue region. Modified molecules, such as pharmaceuticals with peptide or protein extensions, can be allowed to migrate to the region of interest and may be activated to cross-link with the proteins in the target tissue. Alternatively, the complex may be allowed to be taken up by the cell and then activated, preventing it from exiting the cell. For example, a toxic agent used to treat cancer may be deposited in the tumor itself, either intra- or extra-cellularly, thus reducing its rate of elimination from the area to be treated. Active molecules and drugs may be encased in liposomes, viruses or other vehicles, and deposited in an area to provide a slow-release mechanism, and/or release in a desired region.

Formulations or compositions of the present invention may be designed to inductively couple with ambient radiofrequency energy. Such formulations or composition include transducer materials, such as electrical conductors, semiconductors, or ionomers, which can be linked to a group of reactant molecules, thereby enhancing the ability of the molecules to interact with the ambient radiofrequency electromagnetic energy, and so proceed with the desirable alteration or biochemical reaction. Examples of such ionomers include without limitation styrenated ethylene-acrylic acid copolymer or its salts, sulfonated polyesters and their salts, sulfonated polystyrene and its salts and copolymers, polyacrylic acid and its salts and copolymers, hydroxy/carboxylated vinylacetate-ethylene terpolymers, functionalized acrylics, polyesters, urethanes, epoxies, alkyds, latex, gelatin, soy protein, casein and other proteins, alginate, carrageenan, starch derivatives, ionic polysacharides, and the like, sodium polystyrenesulfonate. Additionally, proteins such as gelatin, soy protein, casein, and collagen may be used.

The formulations or compositions of the present invention may include microscopic particles of a susceptor which, when coupled to a molecule, become transducers, resulting in an increase in energy in the regions surrounding the particles. Susceptors used as transducer species may comprise matter with a non-zero electrical conductivity such as, but not limited to, electrical conductors, semiconductors or ionomers. They may be ferromagnetic, diamagnetic or paramagnetic. The coupling may occur through direct bonding of the susceptor to the molecule or the susceptor may reside in the medium. In this way, energy is imparted to the molecules themselves, which ultimately can result in acceleration of reactions most likely through the generation of heat or through increased vibration of molecular groups. Increased vibration and the formation of dipoles is thus a form of molecular or atomic migration which is induced by the application of radiofrequency energy in this manner.

The susceptor or transducer may be a metal nanocrystal or metal nano- or micro-particles, semiconducting nano- or micro-particles, magnetic nano- or micro-particles, polystyrene-encapsulated metal particles, buckminsterfullerenes, or liposome-encapsulated metal particles. Metals or metal compounds may be colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, tungsten, or alloys of materials from this group.

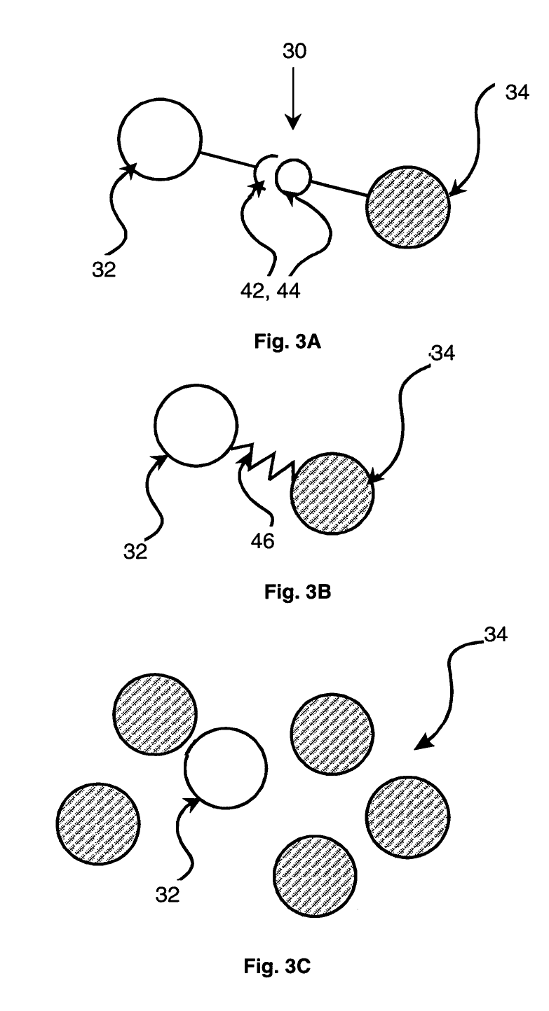

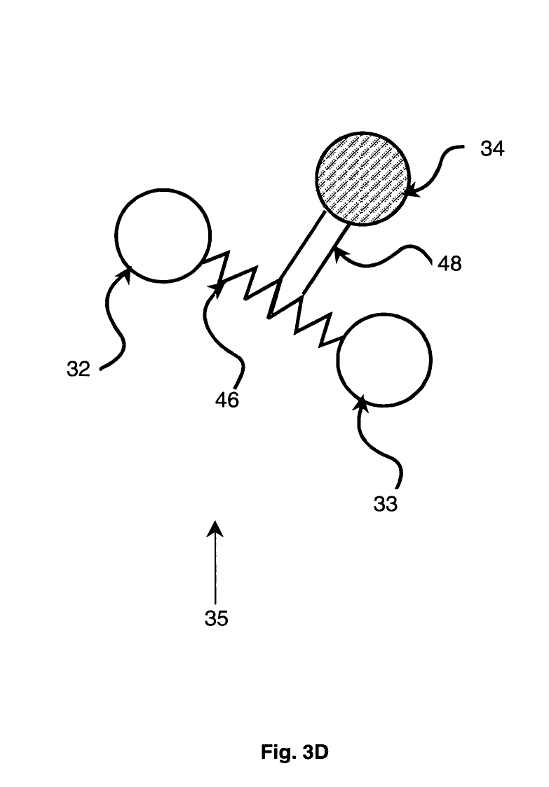

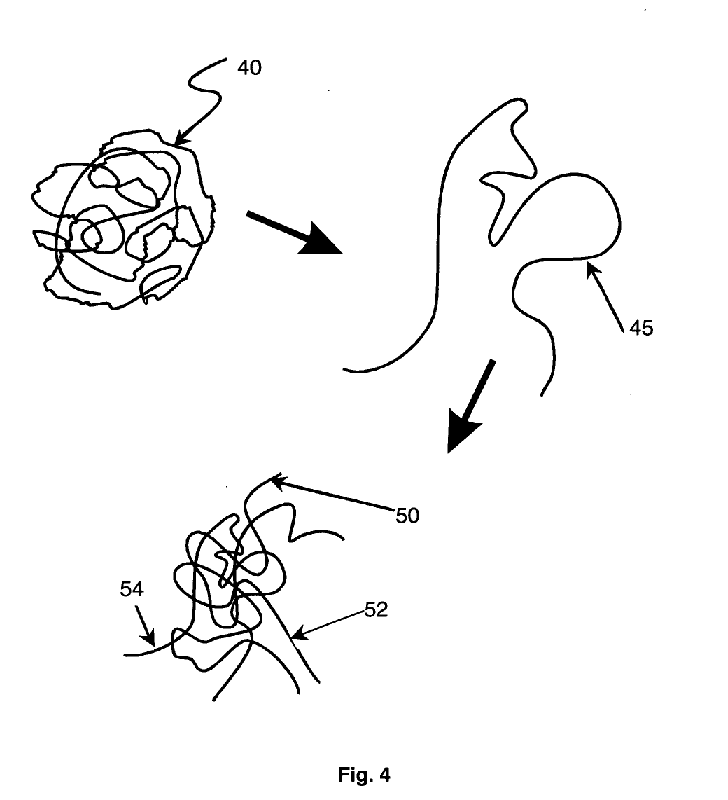

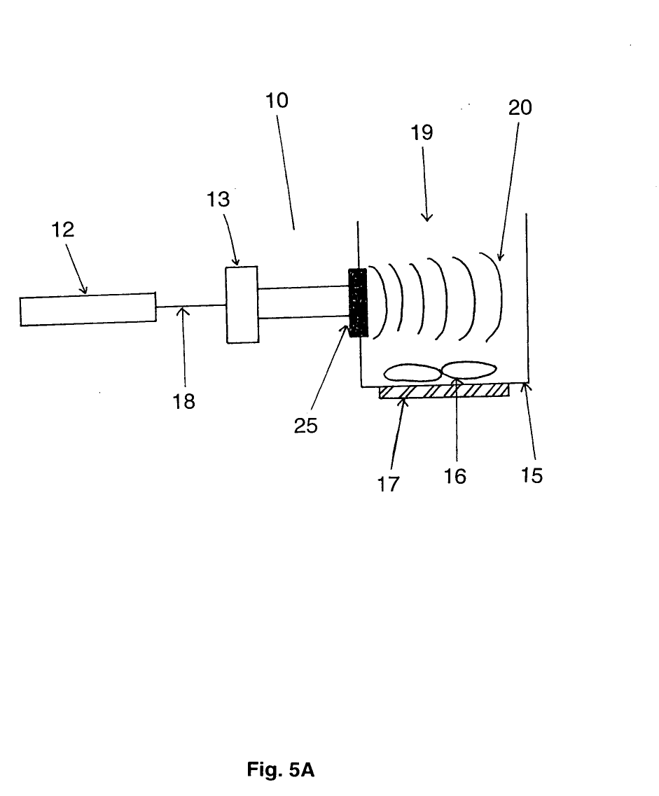

The inductive transducers or susceptors may be positioned in proximity to the reactant using different configurations. In FIG. 3A a reactant 32 and transducer 34 are linked using, for example, avidin and biotin 42,44 bound in no particular order. The resulting molecular species 30 can be prepared from a kit or may be used as components in a kit. During use the molecular species 30 are mixed with appropriate reagents and the avidin-biotin complex 42,44 binds the reactant 32 and transducer 34 in close proximity to one-another so that the transducer 35 can link the ambient radiofrequency energy (not shown) to the reactant 32 to accelerate the reaction. As shown in FIG. 3B, the reactant 32 and transducer 34 are linked with a chemical bond 46. The chemical bond may be covalent or ionic.

Alternatively, FIG. 3C shows the reactant 32 and transducer 34 are not chemically linked, but are in proximity with one-another through stochastic processes such as diffusion. In another configuration, FIG. 3D demonstrates how the inductive transducer 34 is positioned proximate to reactants 32,33 to controllably break the chemical bond 46 between them. The transducer 34 is bonded to the dimer 35 formed by chemically-linked reactants 32,33 with the same bond 46 (not shown) or a different bond 48. Upon activation of the transducer 34, the bond 46 in the dimer 35 is broken.

Additionally, proteins coupled with metal particles or susceptors, whether attached or in the surrounding medium, undergo increased migration in the presence of an alternating magnetic field. This at least in part may be mediated by the generation of heat arising from hysteresis. Migration of proteins in this case most likely involves at least partial denaturation of these and nearby proteins, enabling entire proteins or just strands to migrate and intertwine with one another. Upon renaturation, the individual strands become tightly intertwined, resulting in a type of adhesion or bonding.

The protein-susceptor combination may be used to connect membrane structures such as those in liposomes or in living cells. In the case of liposomes, modifications are made to the formulation to include protein and susceptor in the liposomal membrane. Upon activation, the proteins in the membranes cross-link to form aggregates of one another, and of the liposomes. By controlling time and energy applied, different sizes of these multi-vessicular liposomes may be formed. Such multi-vessicular liposomes are useful in deposition drug delivery as they deposit in a region and are slow to dissolve or resorb, thus resulting in slow, sustained release of the contents.

Clinical applications of either single or multi-vessicular liposomes also include filling the carrier with a pharmaceutical substance and allowing it to localize in a particular region of tissue. Activation of the modified liposome results in cross-linking of the membrane proteins to proteins present in the membranes of tissue cells of the region. Thus, the liposomes became deposited on the tissue of choice.

The present invention provides a method of accelerating a chemical reaction by applying electromagnetic or mechanical energy to the reaction mixture. The energy added to the reaction may be electromagnetic energy or mechanical energy. Examples of electromagnetic energy are radiant energy, radiofrequency energy, microwave energy or magnetic energy. The electromagnetic energy may be generated by a source which provides radiant energy with wavelength from about 200 nm to about 20,000 nm. The electromagnetic energy may have a frequency in the region of the electromagnetic spectrum of about 100 kHz to about 40 GHz. Alternatively, radiofrequency energy may be added inductively to the reactants. Mechanical energy may be a pressure wave. Preferably, the chemical reaction is a catalyzed reaction, such as by an enzyme, and may be either a liquid-phase reaction or a solid-phase reaction.

In the present invention, radiant energy is provided to an enzyme catalyzed reaction mixture such that energy, in the form of heat, is delivered locally in the immediate environment of the molecule, thereby resulting in thermal acceleration of the rate of reaction. This local heating effect is only transient, on the order of less than microseconds, in the case of a pulsed laser. However, it is of sufficient length to result in an increase in the frequency at which these molecules reach their transition state. In the presence of a catalyst, this transition state is stabilized and the reaction proceeds. Specifically, the use of short, high-energy pulses of radiant energy results in little heating of the surrounding medium. The methods provided herein may be used to enhance a polymerase chain reaction by applying energy to the reaction. Such method can also be used for enhancing an enzyme linked immunoassay reaction.

The methods provided herein can increase the rate at which a group of molecules reaches a different molecular configuration from initial configuration, such as a molecular configuration present during a transition state in a biochemical reaction by applying energy to the molecules. Preferably, the energy is electromagnetic energy or mechanical energy. Representative examples of electromagnetic energy include radiofrequency energy and microwave energy and a representative example of mechanical energy is a pressure wave. Still preferably, the different molecular configuration is a transition state in a chemical reaction, preferably, a catalyzed chemical reaction.

Application of electromagnetic energy can introduce heat to molecules, such as a proteins, and cause denaturation and aggregation. As demonstrated in FIG. 4, heat applied to the protein 40 causes it to unfold or denature to a different configuration 45. The denaturated protein 45, in the presence of other denaturated molecules 52 and 54, of the same or different type, tend to aggregate, thus effecting a bond.

The present invention further describes a method whereby a radiofrequency electromagnetic field is used to inductively transfer energy to reactants thereby accelerating a biochemical reaction. One or more of the reactants taking part in the biochemical reaction may have a molecular or macroscopic absorbing species linked to it, or in close proximity to it, for the purpose of enhancing the transduction of energy from the said electromagnetic field to the reactants. Optionally, the reactants may be proteins and the molecular transducer may be an ionomer and the macroscopic transducer may be a metallic nanocrystal or particle, as described supra.

This reaction has multifold beneficial uses, e.g., molecular species can fuse to one another, an artificial or naturally occurring membrane can be modified to increase permeability or an active drug moiety can be released from a pro-drug. For example, it is known that the efficacy of chemotherapeutic drugs in inducing lethal damage to malignant cells increases with the increasing time that the drug is present adjacent to or within the cells. An ongoing problem in cancer therapy is getting malignant cells to retain chemotherapeutic drugs.

A novel and potentially powerful form of cancer therapy would involve the in situ inductive biomolecular alteration or activation of a chemotherapeutic drug/magnetic particle conjugate (6) which would serve to make the cancerous tissue retain the drug. Migration of the altered drug from the desired site of action would be minimized. Without being limited to such theory, this decreased migration may be the result of direct binding of the altered molecule to another species or through an alteration of its mobility characteristics, for example.

The present invention further describes a device comprising a reaction vessel where radiant energy may be supplied in such a way that reactants within the vessel are exposed to a free energy increase in the system which causes increased vibration or rotation in molecular groupings, or causes electrons to shift to excited states making the molecules more reactive. A catalyst present in or attached to the reaction vessel in such a way that the reactants are in contact with the catalyst, will then be in position to stabilize transition states of the reactants, thereby improving the chances that the reaction will proceed forward. The device may be used to accelerate or enhance a chemical reaction such as a polymerase chain reaction or an enzyme linked immunoassay reaction. Additionally, the device may be used to increase the rate at which a group of molecules reaches a different molecular configuration from initial configuration.

Such device may comprise a radiant energy source for heating the reaction, a microvessel containing the reactants and a cooling chamber. Optionally, a catalyst can be included. The vessel alternates between heating and cooling cycles allowing standard PCR reaction to proceed. Alternatively, an optional reaction chamber, without the energy absorbing target/transducer, may be used to directly delivery energy to the reactants for either local heating or for increasing the energy state of the reactants thereby increasing the frequency at which the reactants reach transition state.

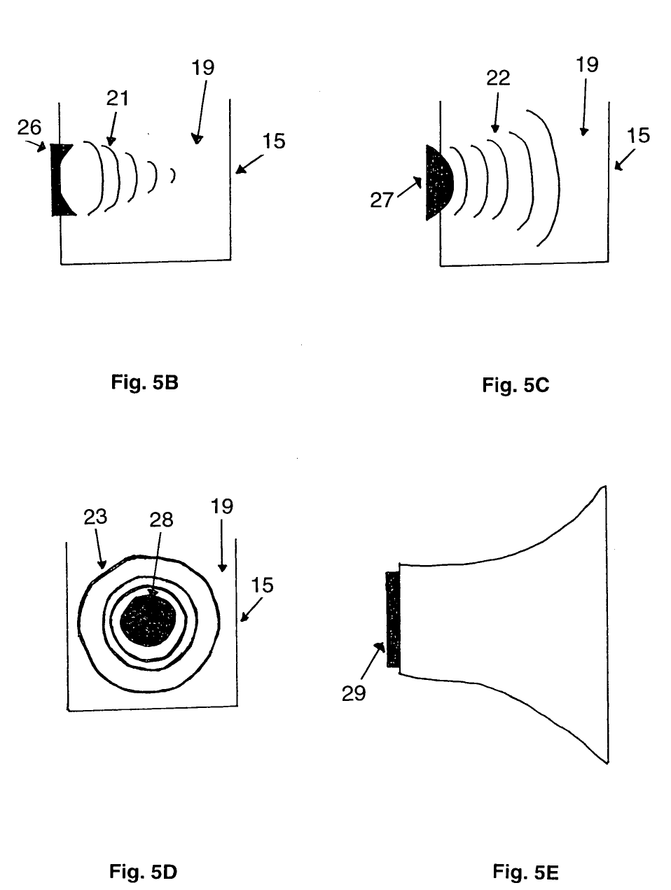

As shown in FIG. 5A, the device 10 comprises a pulsed laser source 12, expanding/focusing optics 13, radiant energy absorbing target/transducer 25, a reaction chamber 15 containing stir bars 16, and a heater/cooler 17 in contact with the reaction chamber 15. The pulsed laser source 12 generates radiant energy in the form of a laser beam 18 which is expanded and focused by the optics 13. The laser beam 18 is absorbed by the target/transducer 25 and transduced into propagating pressure waves 20 in the reaction chamber 5 to interact with a reaction mixture 19. Optionally, the reaction mixture 19 may be stirred so reactants interacting with the propagating pressure waves 20 do so substantially uniformly. Additionally, the heater/cooler 17 may heat/cool the reaction mixture 19 to further promote the reaction.

FIGS. 5B-5E demonstrate that different shaped radiant energy absorbing targets within the walls of the reaction chamber 15, or inside the reaction solution, produce different pressure waves. FIG. 5B shows a target/transducer 26 within the wall of the reaction chamber 15, as in FIG. 5A, having a concave face presented to the reaction mixture 19. The target/transducer 26 transduces the laser beam (not shown) into pressure waves 21 that decrease as they propagate. Alternatively, FIG. 4C shows a target/transducer 27 within the wall of the reaction chamber 15, as in FIG. 5A, with a convex face presented to the reaction mixture 19. The target/transducer 27 transduces the laser beam 18 into pressure waves 22 that increase as they propagate.

FIG. 5D demonstrates that the target/transducer 28 may be in the reaction chamber 15 with the reaction mixture 19. The pressure waves 23 propagate uniformly throughout the reaction mixture 19.

FIG. 5E depicts a target/transducer 29 which may be may be placed in the wall, may be part of the wall, or may be in the reaction chamber.

The reaction vessel may contain at least one reactant and one enzyme that reacts in a step-wise manner to generate products. Radiant energy of a certain wavelength and energy is delivered to the reaction vessel in such a way that the reactants are activated so the first step catalytic reaction proceeds at a faster pace. A second wavelength, and/or energy is then applied to the reaction such that a second step, which requires a different energy of activation, can then take place. These steps may be repeated for cyclic reactions.

For example, in the reaction catalyzed by the enzyme DNA polymerase, a sample of chromosomal or other DNA is present in the reaction chamber with an oligonucleotide primer and the catalyst. In the first step, laser radiation is added to the chamber to increase the energy of the reaction such that the strands of the DNA are denatured and separated either locally or for the greater length of the molecule. When the laser energy is removed, either completely or in between pulses, the strands of DNA will then re-anneal. In the presence of the oligonucleotide primer which matches part of the sequence of a strand of DNA, the primer will compete for binding sites to the strand of denatured DNA. If the primer is in excess relative to the concentration of the sister DNA strand, more primer will bind as opposed to strands re-annealing. In the presence of DNA polymerase, additional nucleotides will be added to the end of the primer such that a new DNA strand is produced. Imparting radiant energy to the reaction chamber will increase the level of activation of the reactants in this case, and the reaction will proceed at a faster rate.

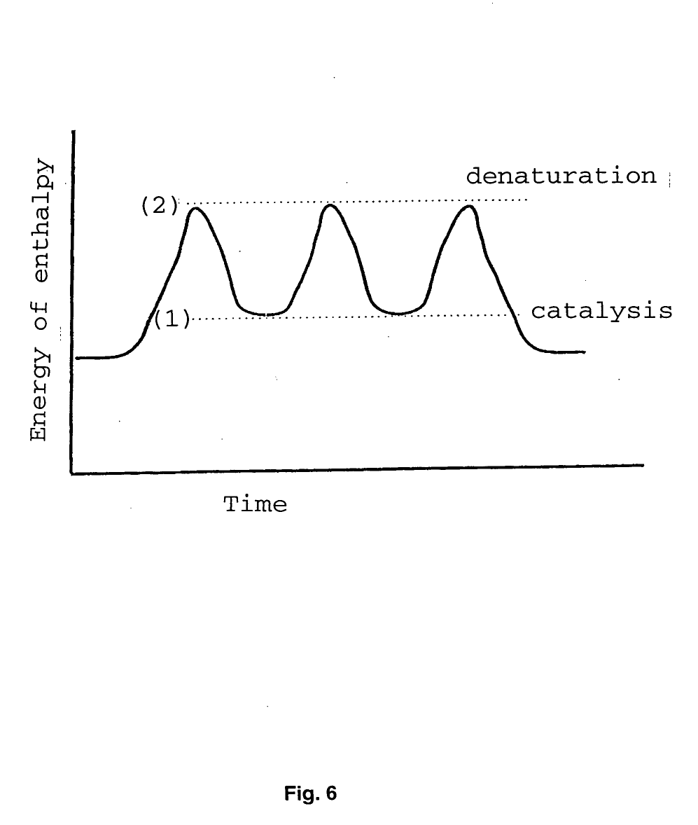

Thus, there are two opportunities to use radiant energy in this reaction. A certain wavelength and sufficient energy is used to first denature the DNA. When the energy is removed or reduced, competitive binding of the oligonucleotide may take place. In the catalytic step, the same or different wavelength radiation will be used, but this time a lower energy that will not denature the double stranded DNA, will then accelerate the rate of the DNA polymerase reaction. By cycling the two wavelengths and/or energies, the reaction may be repeated multiple times (FIG. 6).

In yet another embodiment of the present invention, there is provided a device for inductively transferring energy to reactants, in vivo or in vitro, thus accelerating a biochemical reaction. The devise may comprise one or two antennae. The antenna may be one or more coils of electrical conductor. The electrical conductor is a solid wire or hollow tubing. The induction coils may be a single loop coil, a double loop coil or a multi-loop coil, such as a solenoid, and may be positioned adjacent to and proximate to the reaction vessel. Alternatively, the reaction vessel may be positioned within the induction coil(s). An optional second coil can increase field strength and/or improve field uniformity. The magnitude of the induction field is typically stronger within the turns of the induction coils in contrast to the induction field from the face of an induction coil or antenna.

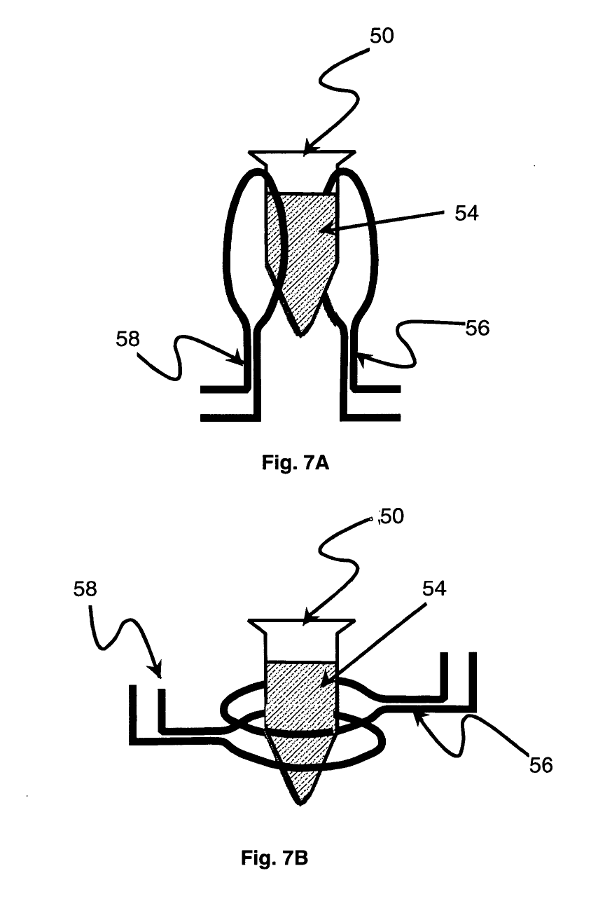

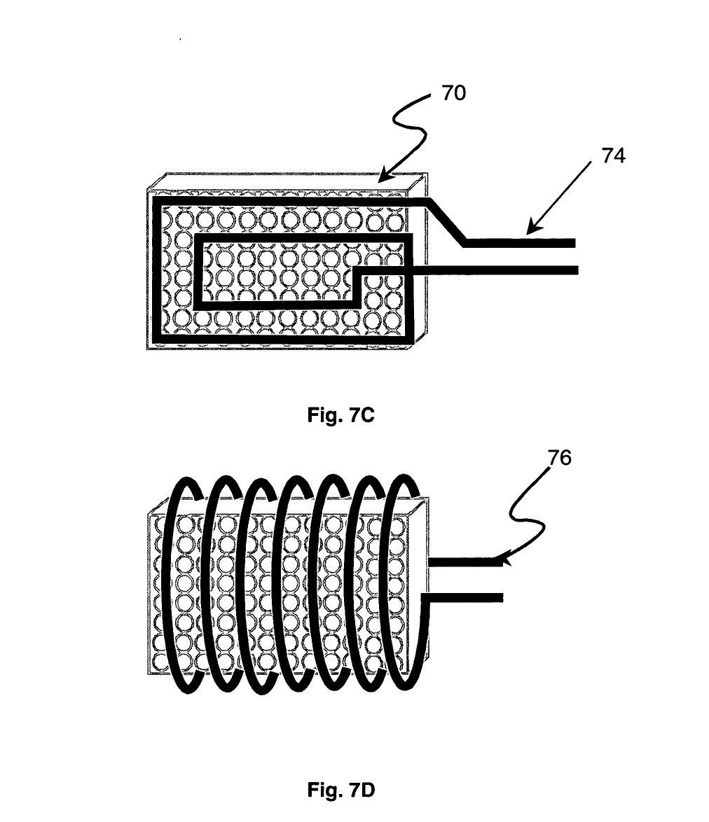

In FIG. 7A, an antennae 56 comprising a single-turn circular coil is positioned in close proximity to a reaction chamber 50, in which a reaction mixture 54 is placed. The antenna 58 shown positioned in proximity to the reaction chamber is optional. FIG. 7B depicts a similar device where the reaction chamber 50 containing reaction mixture 54 is positioned within the turns of the induction coils 56,58. In FIG. 7C, a multiwell plate 70 is positioned against a flat two-loop coil antenna 74. Alternatively, in FIG. 7D, the multiwell plate 70 is positioned within the turns of a solenoid type coil antenna 76.

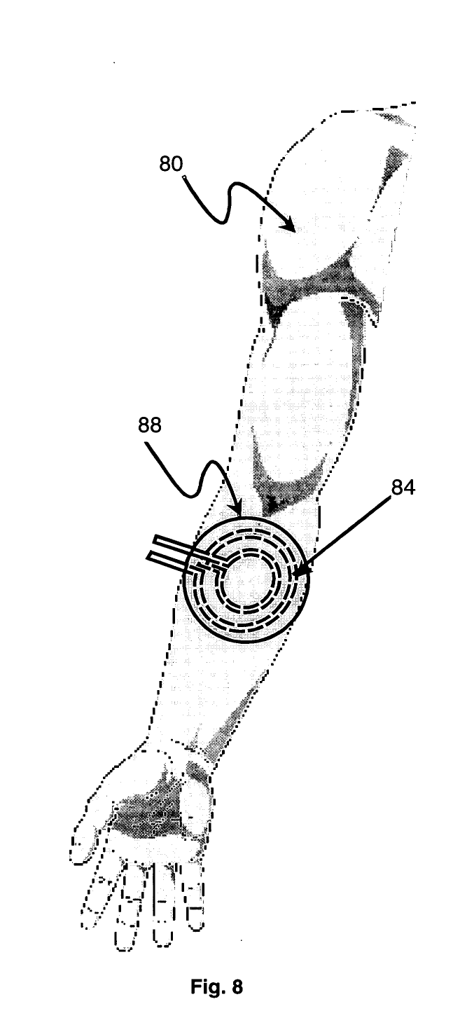

The inductive devices of the present invention can be used to accelerate a biochemical reaction rate in vivo. These devices could be used, inter alia, to induce conformational changes in reactants coupled with transducer species in vivo. FIG. 8 depicts a coil type applicator 88, substantially made out of an electrically non-conducting material, positioned on the arm 80 of a subject. The coil inductor antenna 84 is housed within the applicator.

The following examples are given for the purpose of illustrating various embodiments of the invention and are not meant to limit the present invention in any fashion.

### EXAMPLE 1

Protein Denaturation

A radiofrequency electromagnetic device, operating at 650 kHz, was constructed. Near this frequency, the skin depth in tissue, using conductivity values for canine skeletal muscle at 1 Mhz, is about 205 cm, while for nickel, the value is 14 μm. Two solenoid type coils were constructed using 20G solid copper wire. The coils were encapsulated in a Pyrex sleeve through which low-viscosity mineral oil is circulated as a coolant. Two coils had 200 turns of solid copper wire, formed into a solenoid with a diameter of 2.86 cm and width of 0.95 cm. The magnetic intensity within the bore of the coil was calculated to be greater than 100 kA/m, while at approximately 0.5 cm from a single coil face the intensity is calculated to be maximally 0.15 kA/m. Two coils were electronically connected to the radiofrequency power supply and physically arranged with the bore axis parallel and opposing each other with a gap of about 2 cm between the faces of the coils.

The reactant was ovalbumin at a concentration of 50% w/v albumin in 0.9% saline forming a high viscosity liquid or 75% w/v albumin forming a paste). The transducer species was nickel flake with average particle size of 46 micron, mixed into the albumin solution at 5-10% w/v. The mixture of albumin, saline and nickel comprising the composition, had a highly viscous rheological nature. The composition preparation showed visual evidence, e.g., coagulation and change in opacity, and was warm to the touch after 20-30 seconds when placed between the two solenoid coils with the radiofrequency power supply producing about 210 W.

### EXAMPLE 2

Tissue Fusion

Ex vivo sheep arteries were dissected transversely across the lumen to form sections, or were cut longitudinally to form sheets of tissue. The composition was sandwiched between small sections, e.g., 1 cm², of the tissue sheets and placed between the coils as before. Tissue fusion was apparent by observation. The tissues fused together seamlessly, and it became difficult to tease apart the two sections with forceps. No effort was made to control temperature in these experiments; however, overheating was apparent from a color change in the tissue with longer exposure times, e.g. >45 seconds.

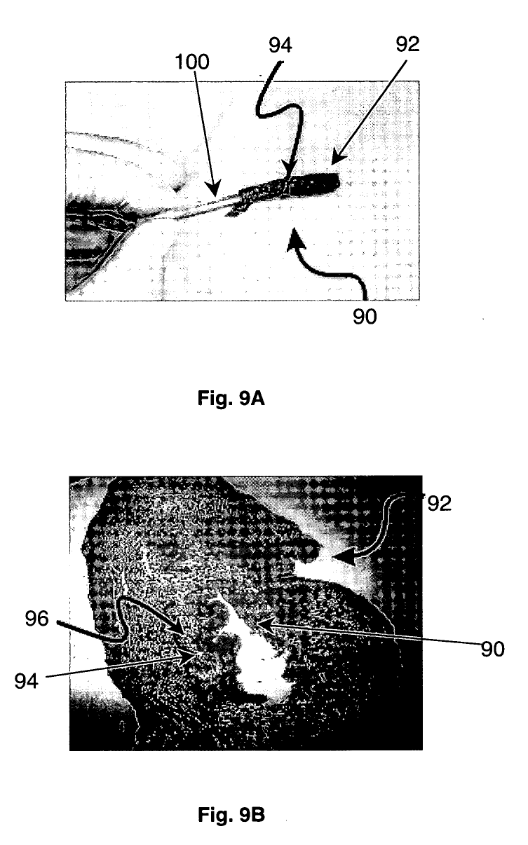

As depicted in FIG. 9A, a composition comprising 5% Ni and 50% albumin (not shown) was placed on the adventitia of one end of a transverse-cut sheep artery 90 and the end of another sheep artery 92 dissected across the lumen was placed over the adventitia of artery 90 and the 200 micron layer of the adhesive fusion composition. A glass rod 100 was used as a support to hold the arteries 90,92 in place. The sample was then positioned between the faces of the opposing coils (not shown) and the sample was exposed for about 30 seconds. The magnetic intensity between the two coils is theoretically estimated to be about 0.3 kA/m. Fusion, or anastomosis, was visually apparent after about ninety seconds and the fused tissue 94 could not be teased apart with forceps without dissection. Tests were repeated five times with equivalent results.

The vessels were placed in 10% formalin, sectioned transversely across the fused area and submitted for histological preparation and staining with hematoxylin-eosin. FIG. 9B shows presence of metallic transducer particles 96 at the interface between the two overlapping sections of arteries 90,92 and delineates the margin of tissue fusion 94.

### EXAMPLE 3

Comparison of Mesh Stainless Steel with Mesh Nickel Compositions

A commercially available induction power-supply (Lepel Corp., Edgewood, NY) modified through the addition of internal capacitors to accept a solenoid coil was used to test different compositions. The device produced an average power of about 100W at a frequency of 400 kHz and a field intensity of 0.3 A/m. The output of the device was coupled into a helical wound coil with an outside diameter of 11 cm made of 11 turns of 1/8 inch copper tubing.

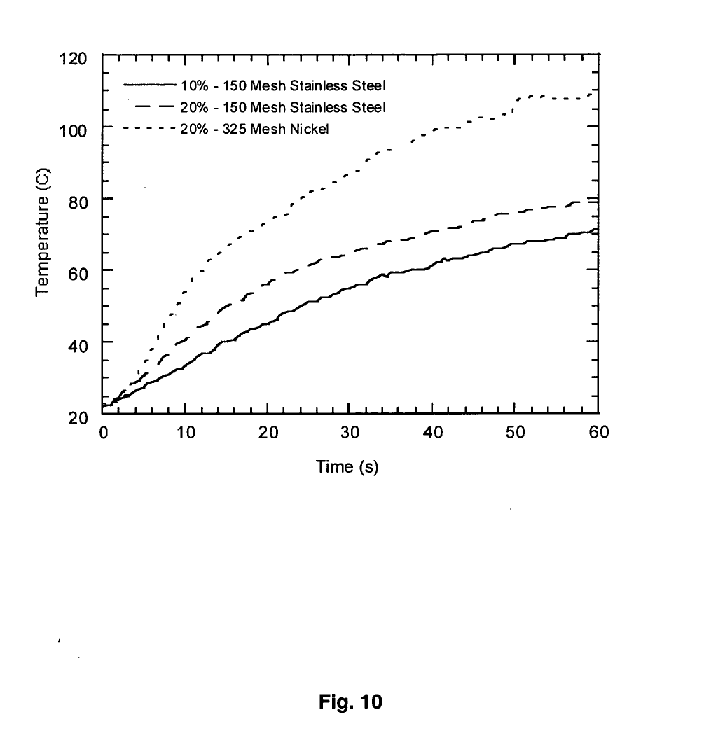

The compositions tested were a formulation of 50% albumin with a tranducer consisting of 10% 150 mesh stainless steel, or 20% 150 mesh stainless steel, or 20% 325 mesh nickel. Each composition was separately positioned within the bore of the coil flush with the surface, and the temperature of the upper surface of the composition was measured with an infrared thermometer (FIG. 10). As expected, nickel heats more efficiently than stainless steel due to its greater magnetic permeability, reaching a threshold temperature of −70° C. within 30 seconds, while stainless steel transducers require double the time.

The following references were cited herein.

1. Flora et al. Unfolding of Acrylodan-Labeled Human Serum Albumin Probed by Steady-State and Time-Resolved Fluorescence Methods, Biophys J., Vol. 75, pp. 1084-1096 (1998).

2. R. Zare. Laser Control of Chemical Reactions, Science, Vol. 279, pp. 1875-1879 (1998).

3. Davies E J. Conduction and Induction Heating. Inst. Elect. Engs. and P. Peregrinus:London (1990).

4. Jordan A. et al. Effects of magnetic fluid hyperthermia (MFH) on C3H mammary carcinoma in vivo, Int. J. Hyperthermia. Vol.13, pp. 587-605 (1997).

5. Hamad-Schifferli K, Schwartz J J, Santos A T, Zhang S and Jacobson J M. Remote Electronic Control of DNA Hybridization Through Inductive Coupling to an Attached Metal Nanocrystal, Nature, Vol. 415, pp. 152-155 (2002).

6. Rudge SR, et al. Preparation, Characterization, and Performance of Magnetic Iron-Carbon Composite Microparticles for Chemotherapy. Biomaterials, Vol. 21, pp. 1411-1420 (2000).

Any patents or publications mentioned in this specification are indicative of the levels of those skilled in the art to which the invention pertains. These patents and publications are herein incorporated by reference to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A composition, comprising:
at least one biomolecule; and
an electromagnetic energy absorbing species associated therewith.

2. The composition of claim 1, wherein said biomolecule is associated with said electromagnetic energy absorbing species via a chemical linker.

3. The composition of claim 2, wherein said chemical linker is an avidin/biotin link.

4. The composition of claim 1, wherein said biomolecule is associated with said electromagnetic energy absorbing species via a chemical bond.

5. The composition of claim 1, wherein said biomolecules form a dimer via a chemical bond, said dimer associated with said electromagnetic energy absorbing species via said same chemical bond or via a different chemical bond.

6. The composition of claim 1, wherein said biomolecules are associated with said electromagnetic energy absorbing species via a physical process.

7. The composition of claim 6, wherein the physical process is diffusion.

8. The composition of claim 1, wherein the biomolecule is a protein, a carbohydrate, or a lipid or a combination thereof.

9. The composition of claim 1, wherein the biomolecule is a pharmaceutical, a biologic, a biomaterial, or a diagnostic or a combination thereof.

10. The composition of claim 1, wherein the electromagnetic energy absorbing species is a susceptor.

11. The composition of claim 10, wherein the susceptor is a metal.

12. The composition of claim 10, wherein the susceptor forms a dipole.

13. The method of claim 1, wherein said electromagnetic energy absorbing species comprises matter with non-zero electrical conductivity.

14. The method of claim 13, wherein said matter is diamagnetic, paramagnetic, or ferromagnetic.

15. The method of claim 13, where said matter is an ionomer, a conducting polymer, an alkali metal, a transition metal, a lanthamide, or a metalloid or a combination thereof.

16. The method of claim 13, where said matter is colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, tungsten, or alloys of materials from this group.

17. The method of claim 16, where said matter is a metal nano- or micro-particle, a semiconducting nano- or micro-particle, a magnetic nano- or micro-particles, a polystyrene encapsulated metal particle, a buckminsterfullerene, or liposome encapsulated metal particles.

18. The composition of claim 1, wherein the electromagnetic energy absorbing species is a dye.

19. The composition of claim 1, wherein the electromagnetic energy absorbed is laser generated or is radiofrequency.

20. The composition of claim 1, wherein the electromagnetic energy is inductively applied to said electromagnetic energy absorbing species.

21. The composition of claim 1, wherein the biomolecule, the electromagnetic energy absorbing species or both undergo a change in state upon application of electromagnetic energy to said composition.

22. The composition of claim 21, wherein the change in state is a cleaved bond or denaturation.

23. The composition of claim 1, further comprising a liposome, said composition incorporated therein.

24. A composition, comprising:
at least one biomolecule; and
a susceptor associated therewith.

25. The composition of claim 24, wherein said biomolecule is associated with said susceptor via a chemical linker.

26. The composition of claim 25, wherein said chemical linker is an avidin/biotin link.

27. The composition of claim 24, wherein said biomolecule is associated with said susceptor via a chemical bond.

28. The composition of claim 24, wherein said biomolecules form a dimer via a chemical bond, said dimer associated with said susceptor via said same chemical bond or via a different chemical bond.

29. The composition of claim 24, wherein said biomolecules are associated with said susceptor via a physical process.

30. The composition of claim 29, wherein the physical process is diffusion.

31. The composition of claim 24, wherein the biomolecule is a protein, a carbohydrate, or a lipid or a combination thereof.

32. The composition of claim 24, wherein the biomolecule is a pharmaceutical, a biologic, a biomaterial, or a diagnostic or a combination thereof.

33. The composition of claim 24, where said susceptor is a metal, a metal nano- or micro-particle, a semiconducting nano- or micro-particle, a magnetic nano- or micro-particles, a polystyrene encapsulated metal particle, a buckminsterfullerene, or liposome encapsulated metal particles.

34. The composition of claim 33, wherein the metal is colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, calcium salts, magnesium salts, or tungsten or alloys thereof.

35. The composition of claim 24, wherein the susceptor forms a dipole.

36. The composition of claim 24, wherein the biomolecule, the electromagnetic energy absorbing species or both undergo a change in state upon application of electromagnetic energy to said composition.

37. The composition of claim 36, wherein the change in state is a cleaved bond or denaturation.

38. The composition of claim 24, wherein said biomolecule(s) comprise at least one protein, said composition further comprising a liposome wherein said protein(s) and said susceptor are incorporated therein.

39. The composition of claim 38, further comprising a pharmaceutical incorporated into said liposome.

40. A method for increasing the energy of biomolecules comprising the steps of
associating the biomolecules with an energy absorbing substance to form the composition of claim 1; and,
applying electromagnetic energy to said composition wherein the electromagnetic energy absorbed by said absorbing species is transferred to said biomolecules thereby increasing the energy thereof.

41. The method of claim 40, further comprising:
accelerating a biochemical reaction having said biomolecules as reactants via said increase in energy.

42. The method of claim 41, wherein said biochemical reaction results in a conformational change in said biomolecules.

43. The method of claim 42, wherein said conformational change is denaturation.

44. The method of claim 42, wherein said biochemical reaction is enzyme catalyzed.

45. The method of claim 44, wherein said biochemical reaction is a polymerase chain reaction or an enzyme-linked immunosorbent assay.

46. The method of claim 40, wherein said biomolecules are in tissue or are in vitro.

47. The method of claim 40, wherein said electromagnetic energy is radiofrequency

48. The method of claim 40, wherein said electromagnetic energy has a frequency from about 100 kHz to 40 GHz.

49. The method of claim 48, wherein said electromagnetic energy has a frequency from about 100 kHz to 10 GHz.

50. The method of claim 40, wherein said electromagnetic energy generates a magnetic field.
